(19) United States
(12) Patent Application Publication
(10) Pub. No.: US 2004/0127413 A1
(43) Pub. Date: Jul. 1, 2004

(54) Enteric keto acid and amino acid salts and their use for preparing medicines

(76) Inventors: Thierry Plouvier, Le Chesnay (FR); Thierry Bouyssou, Baltringen (DE); Serge Biosa, Francueil (FR); Christian Jeanpetit, Bougival (FR); Daniel Kilhoffer, Evreux (FR); Eliane Le Peillet-Feuillet, Boulogne-Billancourt (FR); Francoise Tubery, Igny (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/672,504

(22) Filed: Sep. 26, 2003

Related U.S. Application Data
(63) Continuation of application No. PCT/FR02/01061, filed on Mar. 27, 2002.

(30) Foreign Application Priority Data
Mar. 29, 2001 (FR) 01/04259

(51) Int. Cl. A61K 38/17; A61K 9/24

(52) U.S. Cl. 514/12; 424/471

(57) ABSTRACT

The invention relates to enteric compounds comprising a keto acid, and methods of making and using these compounds in order to treat malnourishment, conditions resulting from malnourishment, and various pathological conditions.

### RELATED APPLICATIONS

This is a continuation application under 35 U.S.C. § 120 of PCT/FR02/01061, filed Mar. 27, 2002, published in French, which claims priority to French Patent Application 01/04259, filed Mar. 29, 2001, both of which are hereby expressly incorporated by reference in their entireties.

### FIELD OF THE INVENTION

The present invention relates to enteric keto acid and amino acid salts, and their use in preparing medicines for treating patients in need, including malnourished subjects or those in hypercatabolism condition. The present invention also relates to treating pathological conditions involving silent neurons, such as defined in PCT application WO 99/47134, which is hereby expressly incorporated by reference in its entirety.

### BACKGROUND OF THE INVENTION

Keto acid and amino acid salts are described in patent WO 99/47134 as being active for the distension model of a colon previously irritated with 1% acetic acid in the rat at a dose of 1 mg/kg. Compounds for this test were orally administered, dissolved in water.

Di-ornithine α-ketoglutarate, also known as ornithine alpha-ketoglutarate or OKG is a salt formed of one molecule of α-ketoglutarate and two molecules of ornithine. Di-ornithine α-ketoglutarate appears to stimulate insulin and growth hormone production and to increase the synthesis of glutamine as well as a number of other amino acids, including arginine and proline (Cynober Curr. Opin. Clin. Nutr. Metab. Care 2:33-37 (1999). Further, di-ornithine α-ketoglutarate has been shown to have anabolic and anticatabolic properties in situations of trauma or stress, to promote wound healing, to improve gut morphology and function and to have the potential to improve immune function.

As a result of its inherent properties, di-ornithine α-ketoglutarate has been used in a wide range of therapeutic applications. For example, it has been used to treat malnourished individuals (Cynober Nutrition 7:313-322 (1991)), to improve nutritive status and quality of life in the elderly (Brocker et al. Age Ageing 23:303-306 (1994)), and to treat burn victims (Donati et al. Clin. Nutr. 18:307-311 (1999)). Di-ornithine α-ketoglutarate is used in therapy under the name CETORNAN® (French patent No. 3 533 M) to improve protein metabolism in malnourished patients.

The therapeutic effectiveness of di-ornithine α-ketogluatrate is limited by the side effects associated with the dosage at which this compound is conventionally administered to achieve beneficial results. At the dosage used in community clinics (5 g twice daily, orally, dissolved in a glass of water), and in hospitals (10 g twice daily, orally and enterally) di-ornithine α-ketoglutarate has the disadvantage of causing diarrhea due to the significant osmolality of the compound (Cynober et al. La Revue du Pratician. 50:1593-1599 (2000)). Another disadvantage of the product is its poor taste in solution.

Thus there exists a need in the art for a compound that is therapeutically effective at doses low enough to prevent unwanted side effects.

### SUMMARY OF THE INVENTION

In certain embodiments the teachings herein relate to enteric compositions comprising at least one compound of the following empirical formula (I):

(X)n₁Y(Z)n₂

wherein X and Z are amino acids, Y is a unbranched keto acid; and n₁ and n₂ represent 0 or 1, wherein said compound is in association with a physiologically stable enteric vehicle. In more specific embodiments, X and Y or Y and Z are in the form of a salt. Additionally, X, Y, and Z can be in the form of a salt. Further embodiments include enteric compositions that are stable at a pH as low as about 1.

In certain embodiments, enteric compositions provided herein can include at least one compound of the empirical formula (I) wherein: n₁ and n₂ are independently 0 or 1, and at least one of n₁ and n₂ is 1; X is a natural amino acid, provided that when n₂=0 then X represents a basic amino acid selected from the group consisting of ornithine, arginine, lysine, and histidine; Y is a keto acid of the following formula (II):

R—CO—COOH

wherein R is an alkyl group or a linear alkane acid with about 1 to about 10 carbon atoms; and Z is a natural amino acid, selected from the group consisting of ornithine, arginine, lysine, histidine, proline and glutamine. In more specific embodiments, R is selected from the group consisting of: —CH₃, —CH₂-CH₃, —(CH₂)₂-COOH, and —(CH₂)₃-COOH.

Further embodiments include compositions including at least one compound from the formula (I) wherein:

n₁=1, and n₂=0 or 1;

X is an amino acid selected from group consisting of ornithine, lysine and arginine;

Y is a keto acid selected from the group consisting of alpha-ketoglutaric acid and alpha-ketobutyric acid; and

when n₂=1, Z is a natural amino acid. In particular aspects, a natural amino acid is selected from the group consisting of ornithine, arginine, proline and glutamine.

Further embodiments relate to compositions including at least one compound from the formula (I) wherein:

n₁=1, and n₂=0;

X is ornithine, and Y is alpha-ketoglutaric acid, or

X is ornithine, and Y is alpha-ketobutyric acid, or

X is arginine, and Y is alpha-ketobutyric acid, or

X is lysine, and Y is alpha-ketobutyric acid, or

X is histidine, and Y is alpha-ketobutyric acid.

Additional embodiments relate to compositions comprising at least one compound from the formula (I) wherein:

n₁=1, and n₂=1; and

X is ornithine, Y is alpha-ketoglutaric acid, and Z is ornithine, or

X is arginine, Y is alpha-ketoglutaric acid, and Z is arginine, or

X is ornithine, Y is alpha-ketoglutaric acid, and Z is glutamine, or

X is ornithine, Y is alpha-ketoglutaric acid, and Z is proline.

In specific embodiments the enteric composition can include the compound diornithine alpha-ketoglutarate.

Enteric compositions described herein can include physiologically stable enteric vehicles selected from the group consisting of enteric microgranules, coated enteric microgranules, enteric nanoparticles or nanospheres, enteric microspheres, enteric microcapsules, enteric granules, coated enteric granules, enteric liposomes, coated enteric liposomes, enteric lyocs, coated enteric lyocs, osmotic pumps with an enteric coating, gums, enteric spheroids, enteric spherical particles, coated enteric spheroids, coated enteric spherical particles, coated enteric tablets, and coated enteric capsules.

In certain embodiments, enteric compositions can be in a form selected from enteric tablets, capsules, sachets and granules.

In further embodiments, the enteric compositions provided herein include a nutritional material. More specifically, the nutritional material can be a food. In particular embodiments, the nutritional material is in a form that is dilutable or dispersible in an aqueous solvent.

In other embodiments, the enteric compositions provided herein are contained within a medicinal drug, wherein said medicinal drug is associated with a pharmaceutically acceptable vehicle. In more specific embodiments, the medicinal drug is in a form that can be orally or enterally administered.

Other embodiments herein relate to enteric compositions comprising at least one compound of the following empirical formula (III):

(X)n₁Y(Z)n₂

wherein X and Z are amino acids, Y is a branched keto acid, and n₁ and n₂ are 0 or 1, wherein said compound is in association with a physiologically stable enteric vehicle. In more specific embodiments, X and Y or Y and Z are in the form of a salt. Additionally, X, Y, and Z can be in the form of a salt. Further embodiments include compositions that are stable at a pH as low as about 1.

More specific embodiments relate to enteric compositions comprising at least one compound of the empirical formula (III) wherein: n₁ and n₂ are independently 0 or 1, and at least one of n₁ and n₂ is 1, X is a natural amino acid, provided that when n₂=0 then X is a basic amino acid selected from the group consisting of ornithine, arginine, lysine, and histidine, and Y is a keto acid of the following formula (II):

R—CO—COOH

wherein R is an alkyl group or a branched alkane acid with about 1 to about 10 carbon atoms.

In certain aspects, R is selected from the group consisting of —CH(CH₃)₂, —CH(CH₃)—CH₂—CH₃, and —CH₂—CH(CH₃)₂.

Further enteric compositions can comprise a compound of the formula (III), wherein:

X is arginine, and Y is alpha-ketoisocaproic acid, or

X is ornithine, and Y is alpha-ketoisocaproic acid, or

X is ornithine, and Y is alpha-keto-beta-methylvaleric acid, or

X is arginine, and Y is alpha-keto-beta-methylvaleric acid, or

X is arginine, and Y is alpha-keto-isovaleric acid, or

X is ornithine, and Y is alpha-keto-isovaleric acid.

The teachings herein also relate to methods of treating a mammal in need of treatment, comprising administering a therapeutically effective amount of an enteric composition provided herein to the mammal. In certain embodiments, it is advantageous to use an enteric composition comprising diornithine alpha-ketoglutarate.

In certain embodiments, the methods disclosed herein can be used on human beings. In particular embodiments, the enteric compositions can be administered orally.

In further embodiments, the mammal to be treated is suffering from a disease or disorder. Specific examples of treatable disorders include pathologies of the digestive tract, pathologies of the biliary ducts, pathologies of the bladder, hemorrhaging proctocolitis, Crohn's disease, gastric ulcers, duodenal ulcers, chronic gastritis, colorectal cancer, gastric cancer, gastroenteritis, intestinal flu, radical ileitis, bladder spasms, vesicle paresis, diarrhea, spasms, constipation and megacolon megarectum.

In additional embodiments the mammal to be treated is in a hypercatabolic state. Hypercatabolic states can be related to burns, scabs, trauma, cardiac impairment, respiratory incapacity, cancer, AIDS, healing of the intestinal membrane or recent surgery, for example.

Mammals that can be treated with the methods herein can also be suffering from conditions including anorexia, gastroparesis, retarded digestive transit, digestive malabsorption, Alzheimer's disease and kidney failure.

In other embodiments, mammals suffering from malnourishment, insulin or growth hormone deficiency, or undergoing healing can be treated with the methods herein.

In certain embodiments, the therapeutically effective amount of the enteric compositions is between about 25 mg to 10 g in a single dose. In more specific embodiments, the therapeutically effective amount of the enteric compounds is between about 100 mg to 5 g in a single or double daily dose.

### BRIEF DESCRIPTION OF THE FIGURES

FIG. 1 is a bar graph demonstrating the effectiveness of diornithine alpha-ketoglutarate microgranules to treat abdominal contractions. The dosages used were 0.1 mg, 0.25 mg, 0.5 mg, and 1 mg.

FIG. 2 is a bar graph comparing the effectiveness of intraduodenal administered (i.d.), and oral administered (p.o.) diornithine alpha-ketoglutarate at preventing abdominal contractions. Both forms were administered at 0.1, 1, and 10 mg/kg.

FIG. 3 is a point graph demonstrating the ability of orally administered diornithine alpha-ketoglutarate to effect weight gain (measured in grams) in malnourished rats. Drugs were administered at pH 6 and pH 1, and dosages of 1 and 3 mg/kg.

FIG. 4 is a point graph demonstrating the ability of orally administered diornithine alpha-ketoglutarate, in a dry enteric composition compared to a dry non-enteric composition, to effect weight gain (in grams) in malnourished rats. The two forms of the drug were tested at a dosage of 1.2 mg/kg p.o.

### DETAILED DESCRIPTION

The teachings herein arise from the unexpected discovery that particular keto acid and amino acid salts are active in the colon distension model and in a nutrition model wherein the pH is neutral. These same salts are generally inactive when tested at pH 1, regardless of the tested dose.

While not being limited to a particular theory, it is believed that when a keto acid and amino derivative salt is orally administered, it passes into a stomach, where without food or drink, the pH is about equal to 1. Traditionally, this acidic environment tends to cause the salt to dissociate into inactive components. In order to compensate for this dissociation, conventional methods increase the dosage of these salts so that part of the non-dissociated product will pass through the stomach barrier and reach its drug target.

In certain embodiments, the teachings herein relate to enteric compositions comprising compounds of the following empirical formula (I):

(X)n₁Y(Z)n₂

wherein X and Z are identical or different amino acids, natural or otherwise, Y is an unbranched linear keto acid, and n₁ and n₂ independently represent 0 or 1. In further embodiments, at least one of n₁ and n₂ represent 1. In other embodiments, the compound occurs in the form of a salt between two constituents X and Y, or Y and Z, or between three constituents X, Y and Z.

In advantageous embodiments, the compounds provided herein are stable in neutral medium and at a pH less than 6. In particular embodiments, the compounds in the above compositions are stable to a pH of about 1.

In some embodiments the compounds of formula (I) generally result from the formation of ionic links between the different constituents X, Y or Z, as opposed to covalent links. Consequently, in certain embodiments, there is no particular significance to the order in which the different constituents of the formula (I) appear. In advantageous embodiments, formula (I) comprises compounds of the empirical formula (X)n₁Y(Z)n₂, as well as those of the structural formula (Z)n₂Y(X)n₁; (X)n₁(Z)n₂Y; (Z)n₂(X)n₁Y; Y(X)n₁(Z)n₂; or Y(Z)n₂(X)n₁.

In particular embodiments, the compositions herein are resistant to acidic medium, and allows for activity by oral administration to be maintained.

Other embodiments of the teachings herein, relate to the reduction of diarrhea induced by the compounds. In more particular embodiments, the compositions provided herein are enteric, thereby allowing for effective treatment at reduced dosages and osmolality.

In further embodiments, a coating can be applied to the enteric compositions provided herein in order to mask the bad taste of the keto acid.

In other embodiments, the enteric compositions provided herein are salts that do not dissociate into dissociation products in the stomach. In more particular embodiments, if dissociation occurs, the enteric salt compounds provided herein dissociate into dissociation products in proportions less than about 20%, and preferably less than approximately 10%, or even less than 20% at a pH about equal to 1. In even more particular embodiments, the enteric compositions provided herein do not degrade into dissociation products at a pH of about 7 to about 1.

In certain embodiments, the dissociation products relate to molecules remaining after the separation of one or more links of the enteric compounds, provided herein. In more specific embodiments, these dissociation compounds can include products such as the salt formed between the keto acid Y and either amino acid X or Z, the keto acid Y itself, or amino acids X and Z, without keto acid Y. In a specific example, possible dissociation products of di-ornithine α-ketoglutarate are ornithine α-ketoglutarate, α-ketoglutaric acid and ornithine.

More particularly, the teachings herein relate to enteric compositions such as defined above, comprising compounds of the empirical formula (I), wherein n₁ and n₂, independently of each other, represent 0 or 1. In more specific embodiments, at least one of n₁ and n₂ represent 1. In still other embodiments, X is a natural amino acid. In further embodiments, when n₂=0, X represents a basic amino acid such as ornithine, arginine, lysine, or histidine, for example.

In other embodiments, Y represents a keto acid of the following formula (II):

R—CO—COOH,

wherein R represents an alkyl group or a linear alkane acid with about 1 to about 10 carbon atoms. In more particular embodiments, Y represents a keto acid of formula (II) wherein R represents either a —CH₃, (the keto acid is pyruvic acid), —CH₂-CH₃, (the keto acid is alpha-ketobutyric acid), —(CH₂)₂—COOH, (the keto acid is alpha-ketoglutaric acid), or —(CH₂)₃—COOH, (the keto acid is alpha-ketoadipic acid).

In still other embodiments, Z represents a natural amino acid, particularly an amino acid selected from among ornithine, arginine, lysine, histidine, proline or glutamine.

In further embodiments, the teachings herein relate to enteric compositions comprising compounds of formula (I) such as defined above, in which Y represents a keto acid selected from alpha-ketoglutaric acid or alpha-ketobutyric acid.

In other embodiments, the enteric compositions, provided herein, are selected from among those of formula (I), wherein n₁=1, and n₂=0 or 1, X represents an amino acid selected from among ornithine, lysine or arginine, and Y represents a keto acid selected from among alpha-ketoglutaric acid or alpha-ketobutyric acid, and when n₂=1, Z represents a natural amino acid, particularly ornithine, arginine, proline or glutamine.

More particularly, the enteric compositions provided herein are selected from among those of formula (I) wherein:

n₁=1, and n₂=0, and

X represents ornithine, and Y represents alpha-ketoglutaric acid, that is, monoornithine alpha-ketoglutarate, or

X represents ornithine, and Y represents alpha-ketobutyric acid, that is, monoornithine alpha-ketobutyrate, or

X represents arginine, and Y represents alpha-ketobutyric acid, that is, arginine alpha-ketobutyrate, or

X represents lysine, and Y represents alpha-ketobutyric acid, that is, lysine alpha-ketobutyrate, or

X represents histidine, and Y represents alpha-ketobutyric acid, that is, histidine alpha-ketobutyrate.

In other embodiments, the enteric compositions, provided herein, are selected from among those of formula (I) in which:

n₁=1, and n₂=1,

X represents ornithine, Y represents alpha-ketoglutaric acid, and Z represents ornithine, that is, diornithine alpha-ketoglutarate, or

X represents arginine, Y represents alpha-ketoglutaric acid, and Z represents arginine, that is, diarginine alpha-ketoglutarate, or

X represents ornithine, Y represents alpha-ketoglutaric acid, and Z represents glutamine, that is, ornithine and glutamine alpha-ketoglutarate, or

X represents ornithine, Y represents alpha-ketoglutaric acid, and Z represents proline, that is, ornithine and proline alpha-ketoglutarate.

In certain embodiments, the enteric composition comprising formula (I) is diornithine alpha-ketoglutarate.

In other embodiments, the enteric composition comprising formula (I), is selected from the group consisting of arginine alpha-ketobutyrate, lysine alpha-ketobutyrate, and histidine alpha-ketobutyrate.

In still further embodiments, the enteric compositions provided herein, comprise compounds of formula (I) in which:

n₁=0 or 1, and n₂=1,

when n₁=1, X represents a natural amino acid, such as ornithine, or arginine, and

Y represents a keto acid selected from among alpha-ketoglutaric acid, or alpha-ketobutyric acid.

Advantageously, the enteric compounds provided herein are in the form of salts between two constituents X and Y, Y and Z, or between three constituents X, Y and Z.

The proportion by weight of the different constituents X, Y and/or Z preferably lies between 0.8 and 1.2, so that the sum of the proportions of each constituent is approximately equal to 2 in a two-component salt (X and Y, or Y and Z), or is approximately equal to 3 in a three-component salt (X, Y and Z). Advantageously, the proportion for each different component is approximately between 0.9 and 1.1.

In certain embodiments, the enteric compounds comprise different constituents that are in equimolar relation, such that the sum of the proportions of each of the components is equal to 2 in a two-component salt (X and Y, or Y and Z), or is equal to 3 in a three-component salt (X, Y and Z).

The enteric compositions provided herein can utilize physiologically stable enteric vehicles selected from standard pharmaceutical enteric preparations. Examples of suitable enteric vehicles include enteric microgranules, and more particularly those selected from among neutral saccharose- and cornstarch-based mediums, and the like.

In other embodiments, the enteric vehicle can be coated enteric microgranules, and more particularly those selected from microgranules sprayed with a film-creating and/or coating agent in solution.

In further embodiments, the enteric vehicle can include enteric nanoparticles. More particularly the enteric nanoparticles can be selected from capsules comprising a polymerized material able to retain the active ingredients by sequestration or adsorption. In certain embodiments, the nanoparticles range from 50 to 300 nm in size. In other embodiments, the nanoparticles are spherically shaped.

In still other embodiments, the enteric vehicle can be enteric microspheres. More particularly the microspheres can be solid. In further embodiments, the enteric microcapsules, and more particularly those comprising an envelope, which is itself solid, contain a liquid, solid, or doughlike substance. The enteric vehicle can also be enteric spheroids and spherical particles of solid fill products with or without excipients.

Enteric vehicles can also include enteric granules, and more particularly those selected from among solid products obtained, for example, from saccharose and/or lactose.

In other embodiments, enteric vehicles can be coated enteric spheroids and spherical particles, from solid fill products with or without excipients obtained by extrusion and/or spheronization. For example, spherical particles sprayed with a film-creating and/or coating agent in solution and another excipient that preserves the quality of the film.

Enteric vehicles can also be coated enteric granules, and more particularly granules sprayed with a film-creating and/or coating agent in solution and another excipient that preserves film quality.

Other examples of enteric vehicles include enteric liposomes, and more particularly spherical vesicles with an aqueous cavity at the center and enclosed with phospholipid bilayers. In certain embodiments these vehicles can be 1 μm or smaller.

Other examples of enteric vehicles include coated enteric liposomes, and more particularly liposomes sprayed with a film-creating and/or coating agent in solution and another excipient that preserves the quality of the film.

Enteric vehicles can also comprise enteric lyocs, and more particularly products obtained by lyophilization of dispersions of vesicles of 1 μm or smaller. Further vehicles can include coated enteric lyocs, sprayed with a film-creating and/or coating agent in solution and another excipient that preserves the quality of the film.

Still other examples include osmotic pumps with an enteric coating, and more particularly those selected from among standard coated tablets comprised of an osmotically active core, a semipermeable membrane and a calibrated orifice in the membrane. Enteric vehicles can also comprise gums such as sterculia, tragacanth, xanthan, arabic, and the like.

Further embodiments can comprise coated enteric tablets, solid fill, with or without excipient, sprayed with a film-creating and/or coating agent in solution and another excipient to preserve the quality of the film.

Still other embodiments include coated enteric capsules, solid fill, with or without excipient, sprayed with a film-creating and/or coating agent in solution and another excipient to preserve film quality.

Advantageously, the compositions may be in the form of enteric tablets, capsules, sachets or granules, as described herein, or in other appropriate forms and packing. These different forms can be obtained according to the methods described in “Coated Pharmaceutical Dosage Forms,” by Bauer, Lehmann, Osterwald and Rothgang, published by Medpharm Scientific Publishers, and in “Pharmacotechnie Industrielle,” by Yves Rossetto, both of which are hereby expressly incorporated by reference in their entireties.

The teachings herein also relate to enteric compositions that include a nutritional material. In certain embodiments, the nutritional material is a food. Advantageously, the nutritional material is in a form that can be diluted or dispersed in an aqueous solvent. In certain embodiments, the nutritional material is in single-dose form containing about 10 or 25 mg up to about 20 g or 50 g of at least one compound of formula (I).

More particularly, the teachings herein relate to the use of a nutritional material for treating subjects in a malnourished condition. Examples of such subjects include: undernourished patients, anorexic subjects, patients with gastroparesis, subjects with slow gastric transit, subjects suffering from digestive malabsorption, subjects undergoing dialysis, or patients with Alzheimer's disease. In further embodiments, the teachings herein can be used to treat persons in a hypercatabolic condition. Hypercatabolic conditions can include, for example, persons with respiratory insufficiency, persons suffering from bedsores, burn patients, cancer patients, AIDS patients, post-operative patients, persons undergoing healing of the intestinal mucosa, persons suffering from multiple traumas, patients with cardiac insufficiency. In further embodiments, the teachings herein relate to treating patients undergoing healing, and patients with stimulated production of growth hormone or insulin.

More particularly, the enteric compositions provide herein relate to the use of a nutritional material, with a daily dosage ranging from about 50 mg to about 40 g.

The teachings provided herein also relate to the use of drugs containing an enteric composition, which can be associated with a pharmaceutically acceptable vehicle. Advantageously, the compositions provided herein are in a form that can be orally or enterally administered. More particularly, the compositions are in a dry form for diluting or dispersing in an aqueous solvent to be orally administered. Depending on the patient and the type and the gravity of the illness to be treated, standard drug dosage may vary from about 10 mg to about 40 g of active ingredient, for example.

In certain embodiments, the compositions described herein can be orally and enterally administered, in single doses of about 25 mg to 10 g of active ingredient per dose. In more specific embodiments the composition is administered from 100 mg to 5 g, in 1 or 2 daily doses. In more particular embodiments, the active ingredient is the compound of formula (I).

The teachings herein also relate to the use of an enteric compositions for preparing a medicine intended to treat malnourished subjects, or animals. The teachings herein also relate to the use of an enteric composition, for preparing a medication used to treat malnourished conditions. More specifically the teachings herein can be used to treat human or animal pathologies involving silent neurons, such as pathologies of the digestive tube, bladder and biliary ducts, and more particularly in the symptomatic treatment of pain associated with these pathologies. Examples of such treatable pain include pain resulting from transit disorders and intestinal discomfort arising from intestinal function disorders (e.g., dyspepsia, irritable bowel and irritable colon syndrome), biliary ducts, hemorrhagic rectocolitis, Crohn's disease, gastric and duodenal ulcers, chronic gastritis, colorectal or gastric cancer, gastroenteritis and intestinal flu, an intestinal pseudo-obstruction or colic, radiation ileitis, digestive or visceral post-operative patients, from diarrhea, spasms, constipation, megacolon, megarectum, bladder spasms, or from vesical paresis.

In certain embodiments, the teachings herein relate to the use of enteric compositions for preparing a medicine that can be administered at a daily dosage of active ingredient ranging from about 1 mg/kg/day to about 1 g/kg/day, preferably 20 to 200 mg/kg/day by oral or enteral administration.

The teachings herein also relate to the use of enteric compositions comprising at least one compound of the following empirical formula (III):

(X)n₁Y(Z)n₂

wherein X and Z are identical or different amino acids, natural or otherwise, Y is a branched keto acid, and n₁ and n₂, independently of each other, represent 0 or 1. In certain embodiments at least one of n₁ and n₂ represent 1. In more particular embodiments, formula (III) is a compound in the form of a salt between two components (X and Y, or Y and Z), or among three components (X, Y and Z). In even more specific embodiments, formula (III) includes a nutrional material, or a medicine, intended to treat malnourished conditions, or human or animal pathologies involving silent neurons.

More particularly, the teachings herein relate to the use of enteric compositions comprising at least one compound of the formula (III), for preparing a nutritional material or medicine intended to treat subjects having malnourished conditions, as provided herein. More specifically these compounds can be used to treat subjects with growth hormone or insulin deficiencies.

The teachings herein also relate to the use of the enteric compositions comprising at least one compound of the formula (III), for preparing a medicine intended for symptomatic treatment of pain associated with pathologies involving silent neurons.

More particularly, the teachings herein relate to the use of enteric compositions comprising at least one compound of the formula (III), for preparing a medicine intended for symptomatic treatment of pain associated with pathologies of the digestive tube, bladder and biliary ducts.

In addition, the teachings herein relate to the use of enteric compositions comprising at least one compound of the formula (III), for preparing a medicine intended for symptomatic treatment of pain as listed above.

In further examples, the teachings herein relate to the use of enteric compositions comprising at least one compound of the formula (III), in which:

n₁ and n₂, independently of each other, represent 0 or 1, at least one of n₁ and n₂ representing 1,

X represents a natural amino acid, provided that when n₂=0 then X represents a basic amino acid such as ornithine, arginine, lysine, or histidine, and

Y represents a keto acid of the following formula (II):

R—CO—COOH

in which R represents an alkyl group or a branched alkane acid with about 1 to about 10 carbon atoms. In more particular embodiments, the keto acid has the formula (II) wherein R is selected from:

—CH(CH₃)₂, (the keto acid is alpha-ketoisovaleric acid), or

—CH(CH₃)—CH₂—CH₃, (the keto acid is alpha-keto-beta-methylvaleric acid), or

—CH₂—CH(CH₃)₂, (the keto acid is alpha-ketoisocaproic acid).

In further embodiments, the teachings herein relate to the use of enteric compositions, comprising at least one compound of the empirical formula (III), in which:

X represents arginine, and Y represents alpha-ketoisocaproic acid, (that is, arginine alpha-ketoisocaproate), or

X represents ornithine, and Y represents alpha-ketoisocaproic acid, (that is, ornithine alpha-ketoisocaproate), or

X represents ornithine, and Y represents alpha-keto-beta-methylvaleric acid, (that is, ornithine alpha-keto-beta-methylvalerate), or

X represents arginine, and Y represents alpha-keto-beta-methylvaleric acid, (that is, arginine alpha-keto-beta-methylvalerate), or

X represents arginine, and Y represents alpha-keto-isovaleric acid, (that is, arginine alpha-keto-isovalerate), or

X represents ornithine, and Y represents alpha-keto-isovaleric acid, (that is, ornithine alpha-keto-isovalerate).

The teachings herein will be further illustrated by the following non-limiting Examples.

### EXAMPLE 1

The following provides a detailed description of the preparation of certain enteric compositions provided herein.

1-Equipment

STREA 1-type Aeromatic® fluidized air bed

Bioblock® peristaltic pump

Memmert® autoclave

Nozzle with 0.8 mm opening

2-Materials

| Item | Supplier |
|---|---|
| T16-18 microgranules | SPCI/Mendell |
| PVP K30 | BASF |
| Eudragit L30D-55 | Rohm Pharma |
| Triethyl Citrate (TEC) | SPCI |
| Bi-osmosed water | |

3-Formulation

An aqueous solution (92.4 mL) containing diornithine alpha-ketoglutarate (4.80 g) and PVPK30 (2.89 g) was sprayed for about 113 minutes onto microgranules (300.1 g) preheated for 10 minutes at temperatures between 30 to 35° C., at a flow of 1 mL/min under pressure of 1 bar.

The ventilation was adjusted to 4 at the outset, then increased to 5. The inlet temperature was 43° C. and outlet temperature was 37° C. Under these conditions, 303.37 g of microgranules were prepared.

4-Coating

The coating was performed in 3 phases:

A) First phase: An aqueous solution (115 mL) containing 268.6 g of Eudragit and 8 g of triethyl citrate was sprayed onto 200 g of the previously obtained microgranules for 189 minutes at a flow of 1.6 mL/min., under pressure of 1.1 bars. Start-up ventilation was set at 4.5, then increased to 5, with an inlet temperature of 35° C., and an outlet temperature of 30° C. Under these conditions, 220 g of microgranules were obtained.

B) Second Phase: An aqueous solution (247.8 mL) containing 462.9 g of Eudragit and 14.5 g of triethyl citrate was sprayed onto 200 g of the microgranules obtained in the first phase for 168 minutes at a flow between 1.8 and 2.6 mL/min., under pressure of 0.8 bar. Ventilation was set at 3.5, at an inlet temperature of 36° C., and an outlet temperature of 28° C. 236 g of microgranules were obtained under these conditions.

C) Third Phase: An aqueous solution identical to that used in the 2nd phase was sprayed onto 200 g of the microgranules obtained in the 2^{nd} phase for 148 minutes under the same conditions as above, though at a flow rate of 2.6 mL/min. Under these conditions, 238 g of coated microgranules were obtained. These microgranules each contained 6.4 μg of diornithine alpha-ketoglutarate, 3.86 μg of PVP, 469.81 μg of Eudragit and 48.62 μg of triethyl citrate.

### EXAMPLE 2

The analgesic properties of the prepared compounds from Example 1 were measured on a colic distension model.

Colic Distension Model

Analgesic activity was studied on a digestive pain model in awake rats. The pain was provoked by distension of the colon using a balloon. Male Sprague-Dawley rats weighing approximately 180 g, given no food since the previous day, were used. Under light Fluothane anesthesia, a rectal probe was inserted 5 cm from the anus and 1.5 mL of 1% acetic acid was injected. 1.5 hours after irritation, a latex balloon (empty diameter 2 mm, 1 cm long) mounted on a polyethylene catheter was inserted into the colon at the irritation site.

The product to be tested, or the placebo vehicle (distilled water), was administered by oral solution at a volume of 1 mL. The treated rat was then placed under observation in a crystallizing dish.

Colon distension was performed 2 hours and 30 minutes after irritation. A fixed volume of 1.5 mL of distilled water was used to inflate the balloon and distend the colon. Colon distension provokes digestive pain evident in the form of abdominal cramps. The number of cramps therefore reflects the intensity of the pain. Colon distension was maintained for 10 minutes, during which abdominal cramps were counted.

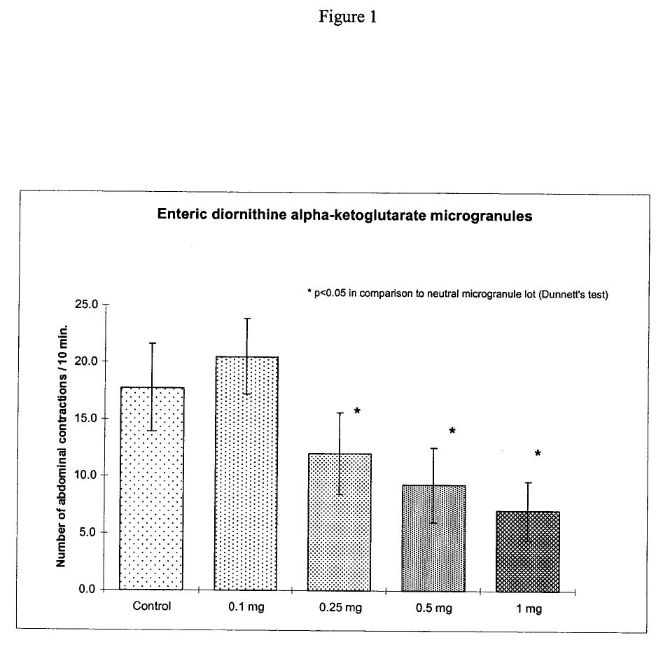

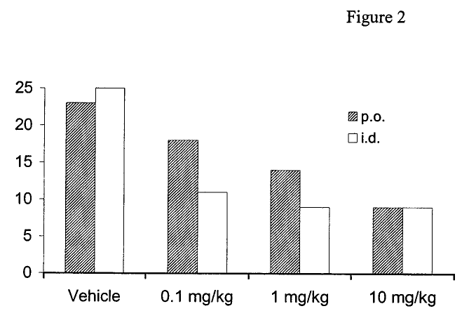

Statistical analysis was carried out using Dunnett's test, which compares a single reference group of vehicle animals (40) to several groups of rats (6 per group) who have received the drugs in question. The significance threshold is 5%. The results are provided in Tables 1 through 5 below, and FIGS. 1 and 2.

By way of illustration, Table 1A demonstrates that Diornithine alpha-ketoglutarate is active at 1, 10 and 20 mg/kg p.o. at pH 6, and inactive at pH 1. Likewise Table 1B demonstrates that monoornithine alpha-ketoglutarate is active at pH 6, and inactive at pH 1. Furthermnore, Table 1C illustrates that arginine alpha-ketobutyrate is active at pH 6, and inactive at pH 1.

TABLE 1A
Visceral Pain Model in the Rat
Effects of diornithine alpha-ketoglutarate at doses
of 1, 10, and 20 mg/kg p.o. tested at pH 6 and pH 1.

| | | Number of abdominal cramps | | |
|---|---|---|---|---|
| | | | Standard | |
| | | Average | deviation | Number |
| Vehicle | | 21 | 4 | 15 |
| Diornithine | 1 mg/kg p.o. pH 6 | 14* | 2 | 4 |
| alpha- | 1 mg/kg p.o. pH 1 | 22 | 4 | 6 |
| ketoglutarate | 10 mg/kg p.o. pH 6 | 13* | 6 | 7 |
| | 10 mg/kg p.o. pH 1 | 20 | 3 | 7 |
| | 20 mg/kg p.o. pH 6 | 11* | 3 | 4 |
| | 20 mg/kg p.o. pH 1 | 19 | 7 | 6 |

*p < 0.05 compared to vehicle

TABLE 1B
Visceral Pain Model in the Rat
Effects of monoornithine alpha-ketoglutarate at dose
of 10 mg/kg p.o. tested at pH 6 and pH 1.

| | | Number of abdominal cramps | | |
|---|---|---|---|---|
| | | | Standard | |
| | | Average | deviation | Number |
| Vehicle | | 20 | 4 | 8 |
| Monoornithine | 10 mg/kg p.o. pH 6 | 10* | 6 | 8 |
| alpha-ketoglutarate | 10 mg/kg p.o. pH 1 | 17 | 3 | 8 |

*p < 0.05 compared to vehicle

TABLE 1C
Visceral Pain Model in the Rat
Effects of arginine alpha-ketobutyrate at dose
of 10 mg/kg p.o. tested at pH 6 and pH 1.

| | | Number of abdominal cramps | | |
|---|---|---|---|---|
| | | | Standard | |
| | | Average | deviation | Number |
| Vehicle | | 19 | 3 | 10 |
| Arginine | 10 mg/kg p.o. pH 6 | 9* | 3 | 10 |
| alpha-ketobutyrate | 10 mg/kg p.o. pH 1 | 15 | 9 | 10 |

*p < 0.05 compared to vehicle

Table 2A demonstrates that after a mixture alpha-ketoglutaric acid and ornithine constituents are dissociated at pH 1, then brought to pH 6, the compounds are inactive. Likewise, Table 2B illustrates that diornithine alpha-ketoglutarate solubilized at pH 1, producing a complete dissociation of the salt, then brought to pH 6, is also inactive. Once dissociated in an acidic environment, the diornithine alpha-ketoglutarate did not reconstitute. Only the mutually salified components are active.

TABLE 2A
Visceral Pain Model in the Rat
Effects of alpha-ketoglutaric acid - ornithine mixture
(ratio 1:2) at doses of 1, 10 and 20 mg/kg
p.o. acidified at pH 1 then neutralized at pH 6.

| | | Number of abdominal cramps | | |
|---|---|---|---|---|
| | | | Standard | |
| | | Average | deviation | Number |
| Vehicle | | 17 | 3 | 8 |
| Alpha-ketoglutaric | 1 mg/kg p.o | 15 | 4 | 8 |
| acid + ornithine | 10 mg/kg p.o | 17 | 4 | 7 |
| | 20 mg/kg p.o | 19 | 6 | 8 |

TABLE 2B
Visceral Pain Model in the Rat
Effects of diornithine alpha-ketoglutarate
tested by oral administration at doses of 1, 10 and
20 mg/kg p.o. acidified at pH 1 then neutralized at pH 6.

| | | Number of abdominal cramps | | |
|---|---|---|---|---|
| | | | Standard | |
| | | Average | deviation | Number |
| Vehicle | | 20 | 1 | 5 |
| Diornithine | 1 mg/kg p.o. | 20 | 1 | 6 |
| alpha-ketoglutarate | 10 mg/kg p.o. | 20 | 4 | 6 |
| | 20 mg/kg p.o. | 21 | 4 | 6 |

Table 3 clearly shows that the enteric form of diornithine alpha-ketoglutarate is active at 1 mg/kg. This activity is equivalent to an intraduodenal dose of 0.1 mg/kg, and considerably greater than for non-enteric microgranules or salt administered in solution.

TABLE 3
Visceral Pain Model in the Rat
Effects of diornithine alpha-ketoglutarate at dose
of 1 mg/kg p.o. administered in enteric microgranules

| | | Number of abdominal cramps | | |
|---|---|---|---|---|
| | | | Standard | |
| | | Average | deviation | Number |
| Vehicle | | 19 | 7 | 8 |
| Neutral microgranules | | 19 | 3 | 8 |
| Diornithine | Coated in neutral | 15 | 6 | 8 |
| alpha-ketoglutarate | microgranules | | | |
| | 1 mg/kg p.o. | | | |
| | Coated in enteric | 7* | 4 | 8 |
| | microgranules | | | |
| | 1 mg/kg p.o. | | | |

*p < 0.05 compared to vehicle

Table 4 also shows the superiority of the enteric form of diornithine alpha-ketoglutarate, with an effective dosage starting as low as 0.25 mg/kg in a visceral pain model.

TABLE 4
Effect of Enteric Diornithine
Alpha-ketoglutarate Microgranules
at Different Doses Administered per os

| | | Number of | | |
|---|---|---|---|---|
| | | abdominal contractions | | |
| | | | Standard | |
| | | Average | deviation | Number |
| Control | | 18 | 4 | 4 |
| Enteric diornithine | 0.1 mg/kg p.o. | 21 | 3 | 4 |
| alpha-ketoglutarate | 0.25 mg/kg p.o. | 12* | 4 | 3 |
| microgranules | 0.5 mg/kg p.o. | 9* | 3 | 4 |
| | 1 mg/kg p.o. | 7* | 3 | 4 |

*p < 0.05 compared to vehicle

Table 5B demonstrates that intraduodenal administration, which avoids gastric acidity, diornithine alpha-ketoglutarate is active starting at a dosage of 0.1 mg/kg, which is a dose ten times lower than the minimum active dose by oral administration (Table 5A). These results are also provided in FIG. 2.

TABLE 5A
Visceral Pain Model in the Rat
Effects of diornithine alpha-ketoglutarate
tested by oral administration at doses
of 0.1, 1, 10 and 20 mg/kg p.o.

| | | Number of abdominal cramps | | |
|---|---|---|---|---|
| | | | Standard | |
| | | Average | deviation | Number |
| Vehicle | | 23 | 5 | 6 |
| Diornithine | 0.1 mg/kg p.o. | 18 | 3 | 7 |
| alpha-ketoglutarate | 1 mg/kg p.o. | 14* | 2 | 4 |
| | 10 mg/kg p.o. | 9* | 4 | 6 |
| | 20 mg/kg p.o. | 8* | 3 | 5 |

*p < 0.05 compared to vehicle

TABLE 5B
Visceral Pain Model in the Rat
Effects of diornithine alpha-ketoglutarate
tested by duodenal administration.

| | | Number of abdominal cramps | | |
|---|---|---|---|---|
| | | | Standard | |
| | | Average | deviation | Number |
| Vehicle | | 25 | 5 | 4 |
| Diornithine | 0.01 mg/kg i.d. | 14 | 6 | 4 |
| alpha-ketoglutarate | 0.1 mg/kg i.d. | 11* | 2 | 6 |
| | 1 mg/kg i.d. | 9* | 3 | 6 |
| | 10 mg/kg i.d. | 9* | 5 | 4 |

*p < 0.05 compared to vehicle

### EXAMPLE 3

Protein Hypercatabolism, Weight Changes And Side Effects

The effectiveness of compounds prepared in Example 1 to act as a nutritional material was measured on a protein hypercatabolism model. Irritation of the colon with 4% acetic acid causes a weight loss in the rat of about 10 to 15%. This chemical stress constitutes a good hypercatabolism model for determining the nutritional effects of the drugs provided herein.

Weight changes were recorded over a 12-day period in female Wistar rats whose colon had been irritated with 4% acetic acid. Female Wistar rats of about 250 g, unfed for 48 hrs., were anesthetized with halothane and their colon irritated with diluted acetic acid (1.5 mL of a 4% solution introduced rectally, 5 cm from the anal border). Upon awakening, the animals—5 per cage—were fed and watered ad libitum. Two days later they were divided into 3 groups of rats of similar weight (rats with weight loss less than 5 g or greater than 30 g were excluded from the study). The test drug or placebo vehicle (distilled water) was orally administered 2 times daily, between 8:00 and 10:00 a.m and between 3:00 and 4:00 p.m. Weight changes were recorded for 12 days, with animal weights measured in grams using a scale.

Statistical analysis was performed using Dunnett's test, which compares a single group of vehicle rats (50) to several groups of rats (ranging from 9 to 19, depending on the group) that have received one of the study drugs. The significance threshold was 5%. Statistical analysis of the diarrhea side effect was performed using a ₁₀₂ ² test that compares the group of rats having received the enteric composition to a group having received the non-enteric composition based on diarrhea compared to non-diarrhea. The significance threshold was 1%.

The drugs were tested by oral administration in both solution and in dry form. For the solution, the drug dosage was at 1 and 3 mg/kg p.o. Drugs were solublized in distilled water, for pH 6 solutions, and in the presence of 1 N hydrochloric acid for pH 1 solutions. A pH meter was used to control pH. Distilled water was used as a placebo vehicle. The drugs in dry form were in an enteric composition at a dosage of 1.2 mg/kg p.o.

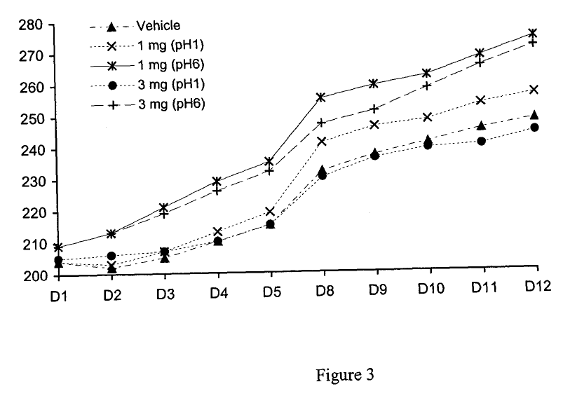

The results for the solution based drugs are as follows. For the rat vehicle group, the initial weight at Day 1 was 204±11 g. Rats reached 248±20 g at Day 12 (N=50). As provided in Table 6 and FIG. 3, at Day 12, rat weight was significantly (p<0.05) higher in the groups having received diornithine alpha-ketoglutarate at 1 and 3 mg/kg p.o. at pH=6. Accordingly, Table 6 and FIG. 3 demonstrate that diornithine alpha-ketoglutarate tested at 1 and 3 mg/kg in a nutrition model is active at pH 6 and inactive at pH 1.

TABLE 6
Malnutrition Model in the Rat
Weight changes (g) in the rat. Effects of
diornithine alpha-ketoglutarate, pH 6 compared
to pH 1, tested at doses of 1 and 3 mg/kg p.o.

| | | | | 3 mg | 3 mg |
|---|---|---|---|---|---|
| Days | Vehicle | 1 mg (pH 1) | 1 mg (pH 6) | (pH 1) | (pH 6) |
| Number | 50 | 18 | 19 | 11 | 9 |
| D1 | 204 ± 11 | 204 ± 8 | 209 ± 10 | 205 ± 7 | 209 ± 10 |
| D2 | 202 ± 14 | 203 ± 10 | 213 ± 16 | 206 ± 11 | 213 ± 15 |
| D3 | 205 ± 17 | 207 ± 13 | 221 ± 19 | 207 ± 11 | 219 ± 19 |
| D4 | 210 ± 20 | 213 ± 17 | 229* ± 18 | 210 ± 14 | 226 ± 20 |
| D5 | 215 ± 22 | 219 ± 16 | 235* ± 18 | 215 ± 16 | 232* ± 18 |
| D8 | 232 ± 22 | 241 ± 11 | 255* ± 19 | 230 ± 21 | 247* ± 20 |
| D9 | 237 ± 21 | 246 ± 11 | 259* ± 19 | 236 ± 21 | 251* ± 19 |
| D10 | 241 ± 20 | 248 ± 13 | 262* ± 18 | 239 ± 20 | 258* ± 20 |
| D11 | 245 ± 20 | 253 ± 11 | 268* ± 19 | 240 ± 21 | 265* ± 21 |
| D12 | 248 ± 20 | 256 ± 11 | 274* ± 18 | 244 ± 20 | 271* ± 21 |

Average weight expressed in grams plus/minus its standard deviation (SD).
*p < 0.05 compared to vehicle

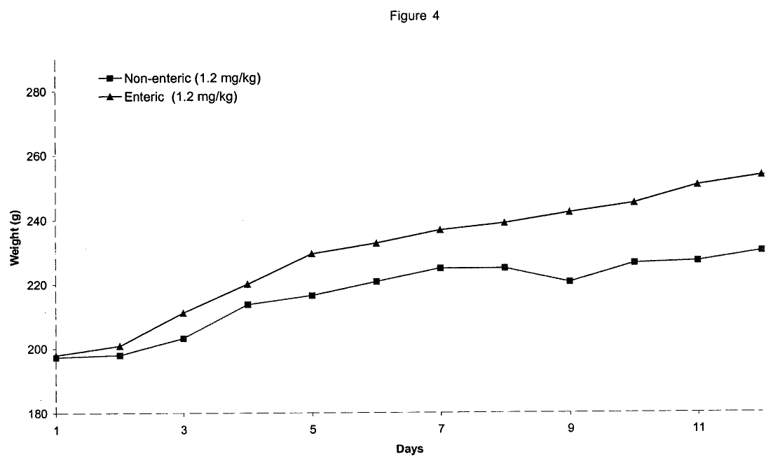

The results for the study using the drug in dry form are provided in Table 7 and FIG. 4. Table 7 and FIG. 4 represent diornithine alpha-ketoglutarate activity for an enteric composition compared to a nonenteric composition in a bioequivalence study at 1.2 mg/kg on a nutrition model. For the group of rats having received the non-enteric composition, the initial weight at Day 1 was 197±7 g. Rats reached 230±28 g at Day 12 (N=10). As provided in Table 7 and FIG. 4, the group of rats treated with the enteric composition had significantly higher weight gain as of Day 5 (p<0.05) compared to the rats administered non-enteric versions of the drug.

TABLE 7
Malnutrition Model in the Rat
Weight changes (g) in the rat. Effects of diornithine
alpha-ketoglutarate, enteric composition compared to
non-enteric composition, tested at a dose of 1.2 mg/kg p.o.

| | Non-enteric | Enteric |
|---|---|---|
| Days | 1.2 mg/kg | 1.2 mg/kg |
| Number | 10 | 10 |
| D1 | 197 ± 7 | 198 ± 6 |
| D2 | 198 ± 11 | 201 ± 8 |
| D3 | 203 ± 14 | 211 ± 12 |
| D4 | 214 ± 17 | 220 ± 11 |
| D5 | 216 ± 20 | 229 ± 12* |
| D6 | 221 ± 17 | 233 ± 12* |
| D7 | 225 ± 21 | 237 ± 10* |
| D8 | 225 ± 26 | 239 ± 12* |
| D9 | 221 ± 32 | 242 ± 12* |
| D10 | 226 ± 29 | 245 ± 11* |
| D11 | 227 ± 31 | 250 ± 11* |
| D12 | 230 ± 28 | 253 ± 10* |

Average weight expressed in grams plus/minus its standard deviation (SD).
*p < 0.05 compared to vehicle

As Table 8 demonstrates, the diarrhea side effect dropped significantly in the group treated with the enteric dry form as compared to the group treated with the non-enteric form, with an alpha risk of 1%. Accordingly, Table 8 shows that an enteric composition compared to a nonenteric diornithine alpha-ketoglutarate composition leads to a highly significant decrease in diarrhea in a nutrition model.

TABLE 8
Malnutrition Model in the Rat
(Diarrhea side effect)

| | Non-enteric | Enteric |
|---|---|---|
| Diarrhea | 84 | 53* |
| Non-diarrhea | 36 | 67 |

*p < 0.01

Although the invention has been described with reference to the above embodiments, it should be understood that various modifications can be made without departing from the spirit of the invention. Accordingly, the invention is limited only by the following claims. All references cited herein are hereby expressly incorporated by reference.

What is claimed is:

1. An enteric composition comprising at least one compound of the following empirical formula (I):
(X)n₁Y(Z)n₂
wherein X and Z are amino acids;
Y is a unbranched keto acid; and
n₁ and n₂ represent 0 or 1,
wherein said compound is in association with a physiologically stable enteric vehicle.

2. The composition of claim 1, wherein said X and Y or Y and Z are in the form of a salt.

3. The composition of claim 1, wherein said X, Y, and Z are in the form of a salt.

4. The enteric composition of claim 1, wherein said composition is stable at a pH as low as about 1.

5. The enteric composition of claim 1, comprising at least one compound of the empirical formula (I) wherein:
n₁ and n₂ are independently 0 or 1, and at least one of n₁ and n₂ is 1;
X is a natural amino acid, provided that when n₂=0 then X represents a basic amino acid selected from the group consisting of ornithine, arginine, lysine, and histidine;
Y is a keto acid of the following formula (II):
R—CO—COOH
wherein R is an alkyl group or a linear alkane acid with about 1 to about 10 carbon atoms; and
Z is a natural amino acid, selected from the group consisting of ornithine, arginine, lysine, histidine, proline and glutamine.

6. The enteric composition of claim 5, wherein R is selected from the group consisting of: —CH₃, —CH₂—CH₃, —(CH₂)₂—COOH, and —(CH₂)₃—COOH.

7. The enteric composition of claim 1, wherein said composition comprises at least one compound from the formula (I) wherein:
n₁=1, and n₂=0 or 1;
X is an amino acid selected from group consisting of ornithine, lysine and arginine;
Y is a keto acid selected from the group consisting of alpha-ketoglutaric acid and alpha-ketobutyric acid; and
when n₂=1, Z is a natural amino acid.

8. The enteric composition of claim 7, wherein the natural amino acid is selected from the group consisting of ornithine, arginine, proline and glutamine.

9. The enteric composition of claim 1, wherein said composition comprises at least one compound from the formula (I) wherein:
n₁=1, and n₂=0;
X is ornithine, and Y is alpha-ketoglutaric acid, or
X is ornithine, and Y is alpha-ketobutyric acid, or
X is arginine, and Y is alpha-ketobutyric acid, or
X is lysine, and Y is alpha-ketobutyric acid, or
X is histidine, and Y is alpha-ketobutyric acid.

10. The enteric composition of claim 1, wherein the composition comprises at least one compound from the formula (I) wherein:
n₁=1, and n₂=1; and
X is ornithine, Y is alpha-ketoglutaric acid, and Z is ornithine, or
X is arginine, Y is alpha-ketoglutaric acid, and Z is arginine, or
X is ornithine, Y is alpha-ketoglutaric acid, and Z is glutamine, or
X is ornithine, Y is alpha-ketoglutaric acid, and Z is proline.

11. The enteric composition of claim 1, wherein the compound is diornithine alpha-ketoglutarate.

12. The enteric composition of claim 1, wherein the physiologically stable enteric vehicle is selected from the group consisting of enteric microgranules, coated enteric microgranules, enteric nanoparticles or nanospheres, enteric microspheres, enteric microcapsules, enteric granules, coated enteric granules, enteric liposomes, coated enteric liposomes, enteric lyocs, coated enteric lyocs, osmotic pumps with an enteric coating, gums, enteric spheroids, enteric spherical particles, coated enteric spheroids, coated enteric spherical particles, coated enteric tablets, and coated enteric capsules.

13. The enteric composition of claim 1, wherein said composition is in a form selected from the group consisting of enteric tablets, capsules, sachets and granules.

14. The enteric composition of claim 1, wherein said composition includes a nutritional material.

15. The enteric composition of claim 14, wherein said nutritional material is a food.

16. The nutritional material of claim 14, wherein said nutritional material is in a form that is dilutable or dispersible in an aqueous solvent.

17. The enteric composition of claim 1, wherein said composition is contained within a medicinal drug, wherein said medicinal drug is associated with a pharmaceutically acceptable vehicle.

18. The medicinal drug of claim 17, wherein said medicinal drug is in a form that can be orally or enterally administered.

19. An enteric composition comprising at least one compound of the following empirical formula (III):
(X)n₁Y(Z)n₂
wherein:
X and Z are amino acids;
Y is a branched keto acid; and
n₁ and n₂ are 0 or 1;
wherein said compound is in association with a physiologically stable enteric vehicle.

20. The enteric composition of claim 19, wherein said X and Y or Y and Z are in the form of a salt.

21. The enteric composition of claim 19, wherein said X, Y, and Z are in the form of a salt.

22. The enteric composition of claim 19, wherein said composition is stable at a pH as low as about 1.

23. The enteric composition of claim 19, comprising at least one compound of the empirical formula (III) wherein:
n₁ and n₂ are independently 0 or 1, and at least one of n₁ and n₂ is 1;
X is a natural amino acid, provided that when n₂=0 then X is a basic amino acid selected from the group consisting of ornithine, arginine, lysine, and histidine; and
Y is a keto acid of the following formula (II):
R—CO—COOH
wherein R is an alkyl group or a branched alkane acid with about 1 to about 10 carbon atoms.

24. The enteric composition of claim 23, wherein R is selected from the group consisting of —CH(CH₃)₂, —CH(CH₃)—CH₂—CH₃, and —CH₂—CH(CH₃)₂.

25. The enteric composition of claim 19, comprising a compound of the formula (III), wherein:
X is arginine, and Y is alpha-ketoisocaproic acid, or
X is ornithine, and Y is alpha-ketoisocaproic acid, or
X is ornithine, and Y is alpha-keto-beta-methylvaleric acid, or
X is arginine, and Y is alpha-keto-beta-methylvaleric acid, or
X is arginine, and Y is alpha-keto-isovaleric acid, or
X is ornithine, and Y is alpha-keto-isovaleric acid.

26. The enteric composition of claim 19, wherein the physiologically stable enteric vehicle is selected from the group consisting of enteric microgranules, coated enteric microgranules, enteric nanoparticles or nanospheres, enteric microspheres, enteric microcapsules, enteric granules, coated enteric granules, enteric liposomes, coated enteric liposomes, enteric lyocs, coated enteric lyocs, osmotic pumps with an enteric coating, gums, enteric spheroids, enteric spherical particles, coated enteric spheroids, coated enteric spherical particles, coated enteric tablets, and coated enteric capsules.

27. The enteric composition of claim 19, wherein said composition is in a form selected from the group consisting of enteric tablets, capsules, sachets and granules.

28. The enteric composition of claim 19, wherein said composition includes a nutritional material.

29. The enteric composition of claim 28, wherein said nutritional material is a food.

30. The nutritional material of claim 28, wherein said nutritional material is in a form that is dilutable or dispersible in an aqueous solvent.

31. The enteric composition of claim 19, wherein said composition is contained within a medicinal drug, wherein said medicinal drug is associated with a pharmaceutically acceptable vehicle.

32. The medicinal drug of claim 31, wherein said medicinal drug is in a form that can be orally or enterally administered.

33. A method of treating a mammal in need of treatment, said method comprising administering a therapeutically effective amount of the enteric composition of claim 1 to the mammal.

34. The method of claim 33, wherein said enteric composition comprises diornithine alpha-ketoglutarate.

35. The method of claim 33, wherein said mammal is a human being.

36. The method of claim 33, wherein said enteric compound is administered orally.

37. The method of claim 33, wherein said mammal is suffering from a disease or disorder.

38. The method of claim 37, wherein said disease or disorder is selected from the group consisting of pathologies of the digestive tract, pathologies of the biliary ducts, pathologies of the bladder, hemorrhaging proctocolitis, Crohn's disease, gastric ulcers, duodenal ulcers, chronic gastritis, colorectal cancer, gastric cancer, gastroenteritis, intestinal flu, radical ileitis, bladder spasms, vesicle paresis, diarrhea, spasms, constipation and megacolon megarectum.

39. The method of claim 33, wherein said mammal is in a hypercatabolic state.

40. The method of claim 39, wherein said hypercatabolic state is related to burns, scabs, trauma, cardiac impairment, respiratory incapacity, cancer, AIDS, healing of the intestinal membrane or recent surgery.

41. The method of claim 33, wherein said mammal is suffering from a condition selected from the group consisting of anorexia, gastroparesis, retarded digestive transit, digestive malabsorption, Alzheimer's disease and kidney failure.

42. The method of claim 33, wherein said mammal is suffering from malnourishment.

43. The method of claim 33, wherein said mammal is suffering from a insulin or growth hormone deficiency.

44. The method of claim 33, wherein said mammal is undergoing healing.

45. The method of claim 33, wherein the therapeutically effective amount of said enteric composition is between about 25 mg to 10 g in a single dose.

46. The method of claim 33, wherein the therapeutically effective amount of said enteric compound is between about 100 mg to 5 g in a single or double daily dose.

47. A method of treating a mammal in need of treatment, said method comprising administering a therapeutically effective amount of the enteric composition of claim 19 to the mammal.

48. The method of claim 47, wherein said mammal is a human being.

49. The method of claim 47, wherein said enteric compound is administered orally.

50. The method of claim 47, wherein said mammal is suffering from a disease or disorder.

51. The method of claim 50, wherein said disease or disorder is selected from the group consisting of pathologies of the digestive tract, pathologies of the biliary ducts, pathologies of the bladder, hemorrhaging proctocolitis, Crohn's disease, gastric ulcers, duodenal ulcers, chronic gastritis, colorectal cancer, gastric cancer, gastroenteritis, intestinal flu, radical ileitis, bladder spasms, vesicle paresis, diarrhea, spasms, constipation and megacolon megarectum.

52. The method of claim 47, wherein said mammal is in a hypercatabolic state.

53. The method of claim 52, wherein said hypercatabolic state is related to burns, scabs, trauma, cardiac impairment, respiratory incapacity, cancer, AIDS, healing of the intestinal membrane or recent surgery.

54. The method of claim 47, wherein said mammal is suffering from a condition selected from the group consisting of anorexia, gastroparesis, retarded digestive transit, digestive malabsorption, Alzheimer's disease and kidney failure.

55. The method of claim 47, wherein said mammal is suffering from malnourishment.

56. The method of claim 47, wherein said mammal is suffering from a insulin or growth hormone deficiency.

57. The method of claim 47, wherein said mammal is undergoing healing.

58. The method of claim 47, wherein the therapeutically effective amount of said enteric composition is between about 25 mg to 10 g in a single dose.

59. The method of claim 47, wherein the therapeutically effective amount of said enteric compound is between 100 mg to 5 g, in a single or double daily dose.
